(19) United States
(12) Patent Application Publication
(10) Pub. No.: US 2008/0119896 A1
(43) Pub. Date: May 22, 2008

(54) METHOD AND DEVICE FOR ANTI-SLEEP APNEA

(76) Inventors: Kwok Shing Thomas Wong, Hong Kong (CN); Xiaoyin Cheng, Hong Kong (CN); Ching Ching Cheung, Hong Kong (CN); Wai Yee Joanne Chung, Hong Kong (CN); Ka Lun Fan, Hong Kong (CN); Yin Ki Lau, Hong Kong (CN); Wai Man Li, Hong Kong (CN); Siu Luen Ng, Hong Kong (CN); Xiaoming Tao, Hong Kong (CN); Harry Zhang, Shanghai (CN)

Correspondence Address: EVAN LAW GROUP LLC, 600 WEST JACKSON BLVD., SUITE 625, CHICAGO, IL, 60661, US

(21) Appl. No.: 11/553,998

(22) Filed: Oct. 28, 2006

(51) Int. Cl. A61N 1/00

(52) U.S. Cl. 607/2; 600/534

(57) ABSTRACT

An anti-sleep apnea device having an elastic abdomen belt optionally with a fabric strain sensor, a first signal-processing unit for processing signals from the fabric strain sensor in response to the change of circumference of the abdomen, two electrodes adapted to be disposed on the user's wrist for releasing electrical pulses to an acupoint of the user, a second signal-processing unit associated with the electrodes, and wireless means for transmitting signals from the first signal-processing unit to the second signal-processing unit. The present invention also discloses a method for managing sleep apnea by applying electrical pulses upon an acupoint of the user.

### FIELD OF THE INVENTION

The present invention relates to a method and a device for anti-sleep apnea particularly utilizing traditional Chinese medicine acupoint theory, bio-signal processing algorithm, and smart textile technology.

### BACKGROUND OF THE INVENTION

There are three main types of sleep apnea, namely obstructive, central and mixed. Obstructive sleep apnea is the blockage of the airway. Central sleep apnea is the failure to control the airway to breathe. Mixed sleep apnea is the combination of obstructive and central sleep apnea. Some common methods for managing sleep apnea are weight management, Continuous Positive Airway Pressure (CPAP) device, oral appliance, surgeries and tongue stimulation.

Weight management may not be an effective approach in the long run. Patients may regain their weight. Also, weight management is not recommended for sleep apnea patients who have craniofacial abnormalities.

Though CPAP device is regarded as a safe and effective alternative for managing sleep apnea, many patients cannot well tolerate the CPAP device. The CPAP devices produce discomfort such as nasal or mouth dryness, psychological discomfort and facial irritation, and increased the number of tossing and turning events during sleep and masks leaking.

With regard to oral appliances, patients may refuse to use them due to the side effects. Oral appliances cause teeth, gum, tongue and jaw discomfort and initial excessive saliva secretion.

Surgery may also produce side effects such as postoperative pain, nasal regurgitation and voice change after receiving surgery.

Electrical stimulation for tongue muscle training may produce discomfort to the users as the electrodes are implanted/placed over the intra-oral area of the patients. The rate of response using the electrical stimulation is variable. Some previous works used an invasive technique to place the electrodes in contact with the hypoglossal nerves. Anesthesia and sterile technique were used. Although positive results were reported with this procedure in obstructive sleep apnea patients, some researchers mentioned that the electrical stimulations increased the number of arousal events while others claimed that the subjects remained asleep during electrical stimulations.

It is known that certain electrically conductive textiles change resistance upon stretching. De Rossi et al. have shown that polypyrrole coated LYCRA shows a fabric strain sensing effect (see De Rossi et al., Mater. Sci. Eng. C, 7(1), 31-35 (1998); De Rossi et al. and De Rossi et al. patent IT 1291473 (1997)). De Rossi et al. believe that this fabric strain sensing effect is due to an increase in the number of contacts between the fibers woven into the fabric when the fabric is stretched.

U.S. Pat. No. 6,360,615 to Smela et al, disclosed the idea of the employment of awearable effect-emitting strain gauge device for producing a variety of smart or intelligent products, including smart garments and accessories.

Prior to the present invention, however, it was unknown that a fabric strain sensing material was ever employed in an anti-sleep apnea device. It was also unknown that releasing electrical pulses to an acupoint along a meridian, particularly, the Lung meridian, relieves sleep apnea.

The above description of the background and cited references are provided to aid in understanding the invention, but are not admitted to describe or constitute pertinent prior art to the present invention.

### SUMMARY OF THE INVENTION

According to one embodiment of the present invention, there is provided an anti-sleep apnea device including two electrodes adapted to be disposed on the user's wrist for releasing electrical pulses to an acupoint of the user; a control unit which comprises an electrical actuator, a high voltage capacitor coupled to the electrical actuator, and a voltage booster for charging the high voltage capacitor; and a switch by which the user can turn on the control unit to send electrical pulses to the acupoint through the electrodes. A preferred acupoint is the Lung Acupoint LU7 (Lieque).

According to another embodiment of the present invention, there is provided an anti-sleep apnea device including a fabric strain sensor adapted to be disposed on a user's abdomen, thorax, or both; a first signal-processing unit for processing signals from the fabric strain sensor in response to the change of circumference of the user's abdomen, thorax or both; two electrodes adapted to be disposed on the user's wrist for releasing electrical pulses to an acupoint of the user; a second signal-processing unit associated with the electrodes; and means for transmitting signals from the first signal-processing unit to the second signal-processing unit. A preferred acupoint is the Lung Acupoint LU7 (Lieque).

In an embodiment, the fabric strain sensor is provided on an elastic belt adapted to be wrapped around the user's abdomen or thorax, and the electrodes are provided on a wristband adapted to be wrapped around the user's wrist.

In an embodiment, the first signal-processing unit includes an amplifier/conditioner and a first micro-control unit. The first micro-control unit is implemented with a software algorithm to monitor the respiratory status of a user using the fabric strain sensor.

In an embodiment, the second signal-processing unit includes a second micro-control unit and an electrical actuator. The second signal-processing unit further includes a high voltage capacitor coupled to the electrical actuator, and a voltage booster for charging the high voltage capacitor.

In an embodiment, the signal transmitting means are RF transmitter and RF receiver. In an embodiment, the anti-sleep apnea device has a set button for presetting the electrical pulses at a preset level of intensity, a LED for displaying the preset level of intensity, or display events of releasing the electrical pulses, or both, and a test button for testing the preset level of intensity of the electrical pulses.

According to another aspect of the present invention, there is provided a method for reducing or eliminating sleep apnea including the steps of detecting the occurrence of sleep apnea with a fabric strain sensor which, in response to the movement of the abdomen, thorax or both associated with occurrence of sleep apnea, generates electronic signals; and transmitting the signals to a pulse-releasing unit thereby effectuating stimulation at a Lung acupoint of the user.

In an embodiment, the method includes the steps of preprocessing the signals of the fabric strain sensor by an amplifier/conditioner, and interpreting the preprocessed signals by a first micro-control unit.

In an embodiment, the method includes the steps of processing the signals from the first micro-control unit by a second micro-control unit, and releasing the electrical pulses by an electrical actuator when the reduction of a calculated index such as mean absolute amplitude or standard deviation of voltage is over a pre-defined threshold.

In an embodiment, the method further includes the steps of providing a high voltage capacitor for storing electrical charges and releasing energy to the electrical actuator, and providing a voltage booster for charging the high voltage capacitor.

In an embodiment, the method includes transmitting the signals from the first micro-control unit to the second micro-control unit by a wireless means.

Although the invention is shown and described with respect to certain embodiments, it is obvious that equivalents and modifications will occur to others skilled in the art upon the reading and understanding of the specification. The present invention includes all such equivalents and modifications, and is limited only by the scope of the claims.

### BRIEF DESCRIPTION OF THE DRAWINGS

Specific embodiments of the invention will now be described by way of example with reference to the accompanying drawings wherein:

FIG. 1 illustrates Lung Acupoints and LU 7 (Lieque) (Chinese Character: [symbol] [symbol] . The scale represents 1.5 inches.

FIG. 2 is a perspective view of an abdomen belt of an anti-sleep apnea device in accordance with an embodiment of the present invention.

FIG. 3 is a perspective view of a wristband of the anti-sleep apnea device in accordance with an embodiment of the invention.

FIG. 4 is a perspective view of the wristband shown in FIG. 3 with the top casing removed.

FIG. 5 is a perspective view of the wristband shown in FIG. 3 with the bottom casing removed.

FIG. 6 is a block diagram of the components of the abdomen belt of the anti-sleep apnea device in accordance with an embodiment of the invention.

FIG. 7 is a block diagram for the components of the wristband of the anti-sleep apnea device in accordance with an embodiment of the invention.

FIG. 8 is a block diagram for other components of the wristband of the anti-sleep apnea device in accordance with an embodiment of the invention.

### DETAILED DESCRIPTION OF THE INVENTION

Reference will now be made in detail to an embodiment of the invention, examples of which are also provided in the following description. Exemplary embodiments of the invention are described in detail, although it will be apparent to those skilled in the relevant art that some features that are not particularly important to an understanding of the invention may not be shown for the sake of clarity.

Furthermore, it should be understood that the invention is not limited to the precise embodiments described below and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims.

In addition, improvements and modifications which may become apparent to persons of ordinary skill in the art after reading this disclosure, the drawings, and the appended claims are deemed within the spirit and scope of the present invention.

It should be noted that throughout the specification and claims herein, when one element is said to be “coupled” to another, this does not necessarily mean that one element is fastened, secured, or otherwise attached to another element. Instead, the term “coupled” means that one element is either connected directly or indirectly to another element or is in mechanical or electrical communication with another element.

In Traditional Chinese medicine there are 365 acupoints that are mapped to 14 major meridian lines: one meridian for each of the 12 inner organs, one meridian along the spine (the governing vessel), and another along the midline of the abdomen (the conception vessel).

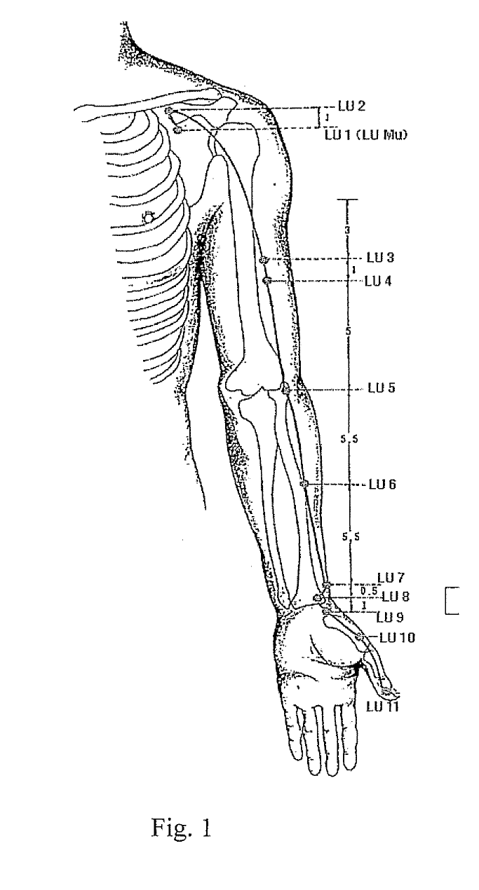

FIG. 1 shows the Lung Acupoints L1-L11. Each lung point has its function in modulating internal body organs and thus achieves the purpose of treating disorders. http://www.acuxo.com/meridianPictures.asp?point=LU11&meridian=Lung. The acupoint LU7, or Lieque, is located 1.5 inches above the wrist, 0.2-0.3 inches deep, on the inside of the arm. The point is in the depression superior to the styloid process of the radius. This point in traditional Chinese medicine is used to treat several disorders of the upper body, including neck stiffness, cough, asthma, and sore throat. While not to be bounded by the theory, it is believed that the LU7 point connects to airway muscle and stimulation of it can immediately improve the condition of airway during sleep apnea.

The present invention applies the traditional Chinese medicine acupoint theory (i.e., specific locations on the body can be stimulated to exert beneficial biological or therapeutic effects), bio-signal processing algorithms and smart textile technology to provide a device for anti-sleep apnea.

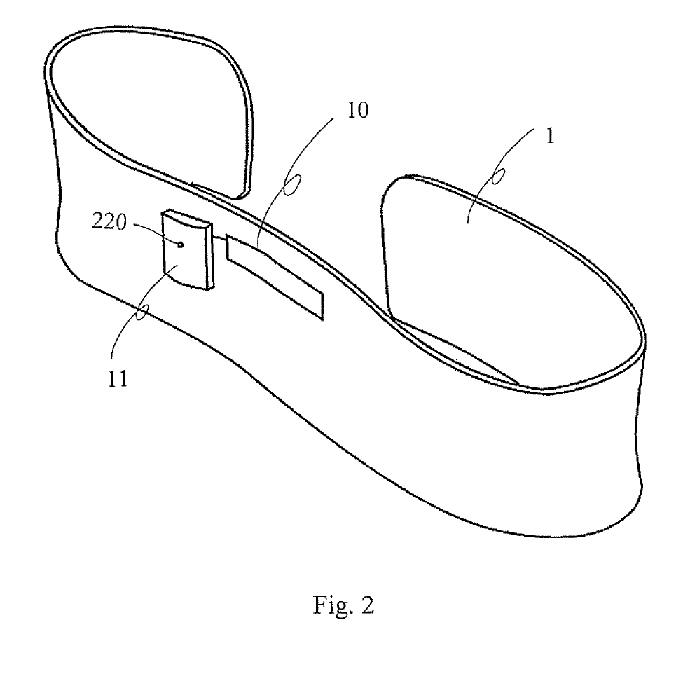

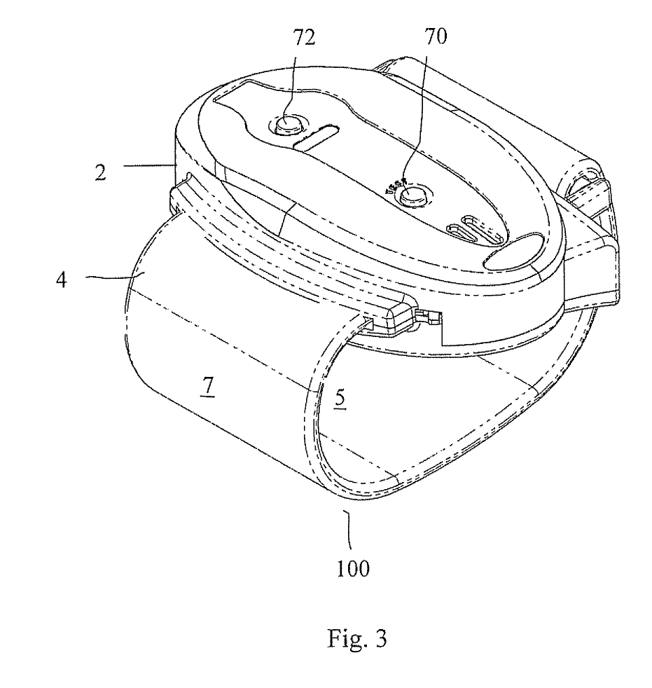

In accordance with the illustrated embodiment of the present invention, the anti-sleep apnea device includes an abdomen belt 1 and a wristband 100 shown in FIGS. 2 and 3 respectively. The abdomen belt 1 is able to promptly detect and indicate the occurrence of sleep apnea. The wristband 100 is able to release electrical stimulation upon the Lieque meridian point (LU 7) of the user in case of the occurrence of sleep apnea.

The abdomen belt 1 and the wristband 100 are non-invasive, portable and light weight since smart textile technology has been utilized to replace some of the traditional electronic sensors and actuators.

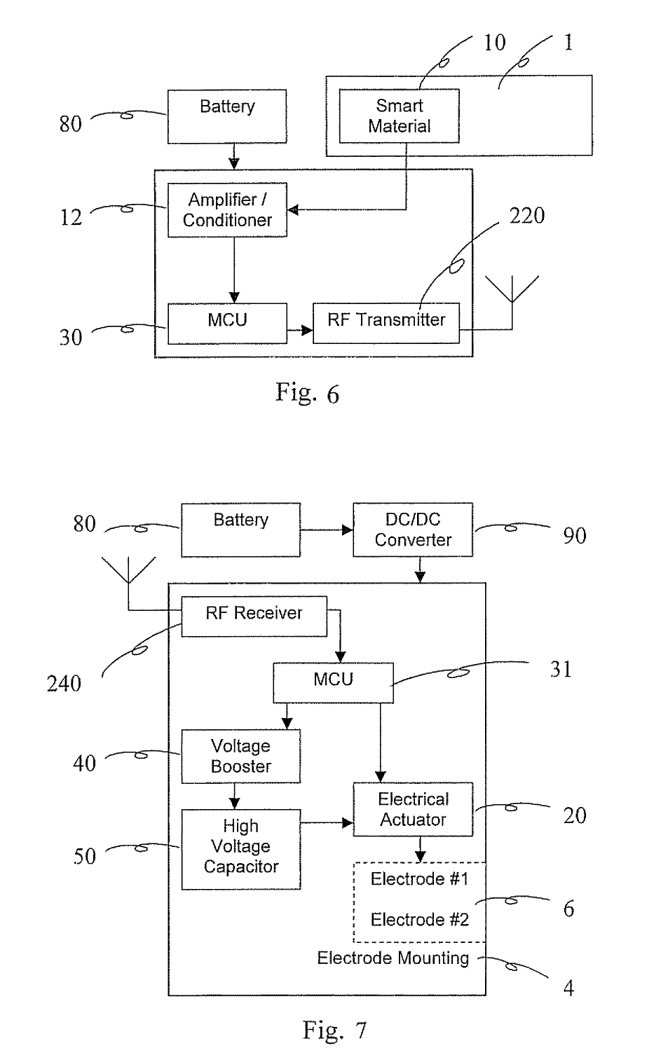

Details of the abdomen belt 1 will now be described with reference to FIG. 2, which is a perspective view of the abdomen belt 1 and FIG. 6 is a block diagram of the components of the abdomen belt 1.

The abdomen belt 1 includes at least one piece of fabric strain sensor 10, an amplifier/conditioner 12, a micro-control unit (MCU) 30, a battery unit 80, and a wireless radio frequency (RF) signal transmitter 220.

For the purpose of this disclosure, “fabric strain sensor” means any conductive fabric whose resistance changes when it is stretched or under strain. The preferred exemplary fabric strain sensor is fabricated according to U.S. patent application Ser. No. 11/222,179 entitled “Method for Conducting Polymer Coating” filed Sep. 9, 2005 and U.S. patent application Ser. No. 11/029,647 entitled “Patterned Electrical Textiles and Array Devices” filed Jun. 1, 2005, which are incorporated herein by reference in their entirety. The fabric strain sensor disclosed therein was produced by coating a conducting polymer onto a substrate. The fabric produced by the method shows improved stability while conductivity and sensitivity are maintained and suitable to be used as strain fabric sensors.

The abdomen belt 1 is made of conventional elastic material (wherein the fabric strain sensor is incorporated) and adapted to be wrapped around the abdomen of the user. Fasteners, such as VELCRO®-type fasteners, or buckles can be employed to fasten the abdomen belt 1 around the abdomen of the user. The process of fabricating the belt is within the ordinary skill possessed by people in the art.

The fabric strain sensor 10, containing conductive material in which the resistance varies in response to applied mechanical strain, is attached to and stretchable along with the elastic abdomen belt 1. The fabric strain sensor 10 is able to indicate the respiratory status of the user. Resistance of the fabric strain sensor 10 will change when a force is applied thereto due to the movements or changes in circumference of the abdomen of the user. In other words, the movements or changes in circumference of the abdomen due to respiration can be measured using the fabric strain sensor 10. The movements or changes in circumference of the abdomen will be measured, for example, in terms of voltage and analyzed in terms of mean absolute amplitude or standard deviation of voltage in every ten seconds.

The change of resistance of the fabric strain sensor 10 is transformed into electronic signal and preprocessed by the signal amplifier/conditioner 12. The preprocessed signal is interpreted by the MCU 30. A software algorithm for sleep apnea detection based on abdominal movement decides the occurrence of sleep apnea on the user. The software algorithm is implemented in the MCU 30. When the occurrence of sleep apnea is detected, the MCU 30 sends a stimulation request signal to the wristband 100 via the RF transmitter 220. The amplifier/conditioner 12, the MCU 30, the battery unit 80, and the RF transmitter 220 are accommodated within a housing 11 on the abdomen belt 1.

Although it has been described that the fabric strain sensor 10 is provided on a belt adapted to be wrapped around the abdomen for detecting the movements or changes in circumference of the abdomen of the user, it is appreciated that the fabric strain sensor 10 may be provided on a belt adapted to be wrapped around the thorax, abdomen or both for detecting the movements or changes in circumference of the thorax, abdomen or both of the user.

Alternatively, the fabric strain sensor 10 may be provided on an article of clothing adapted to be worn by the user during sleep. For example, the fabric strain sensor 10 can be provided on a sleepwear with the fabric strain sensor 10 being attached thereto at the abdomen, thorax or both of the user.

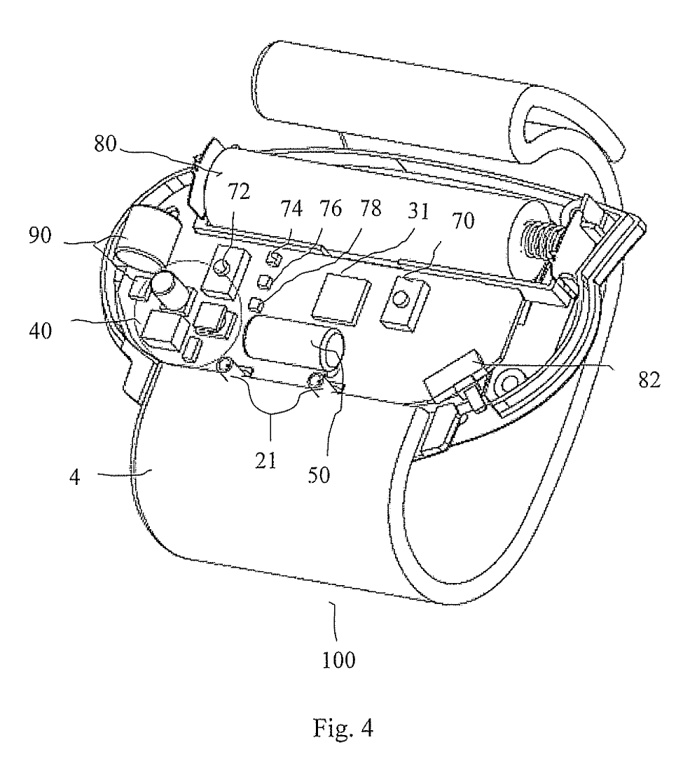

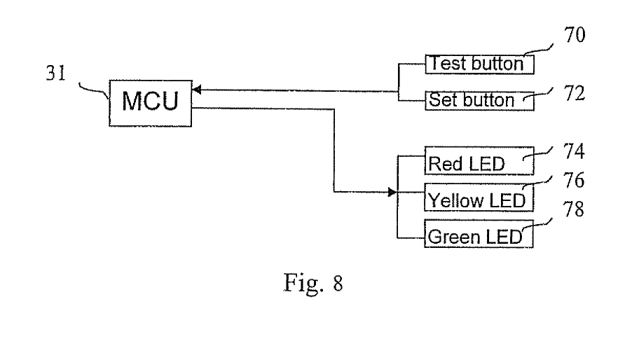

FIGS. 3 to 5 show the wristband 100 of the anti-sleep apnea device in accordance with an embodiment of the invention. FIGS. 7 and 8 are block diagrams of the components of the wristband 100.

The wristband 100 includes a casing 2, a band 4, and two electrically conductive electrodes 6. A wireless RF signal receiver 240, a micro-control unit (MCU) 31 (e.g. ATMEGA48 from ATMEL), a battery unit 80, and an electrical actuator 20 are accommodated within the casing 2.

If the reduction of a calculated index such as the mean absolute amplitude or the variation of its standard deviation is over a pre-defined threshold, the RF receiver 240 will receive the stimulation request signal and the MCU 31 will send a proper electrical stimulation signal to the actuator 20 in the wristband 100 to release electrical stimulation of 8 seconds on the LU7 meridian point.

The user can set the intensities, durations and time of stimulations prior to sleep. The actuator 20 will deliver gentle electrical pulses to the body of the user via the electrically conductive electrodes 6. No conductive gel is applied on the skin surface.

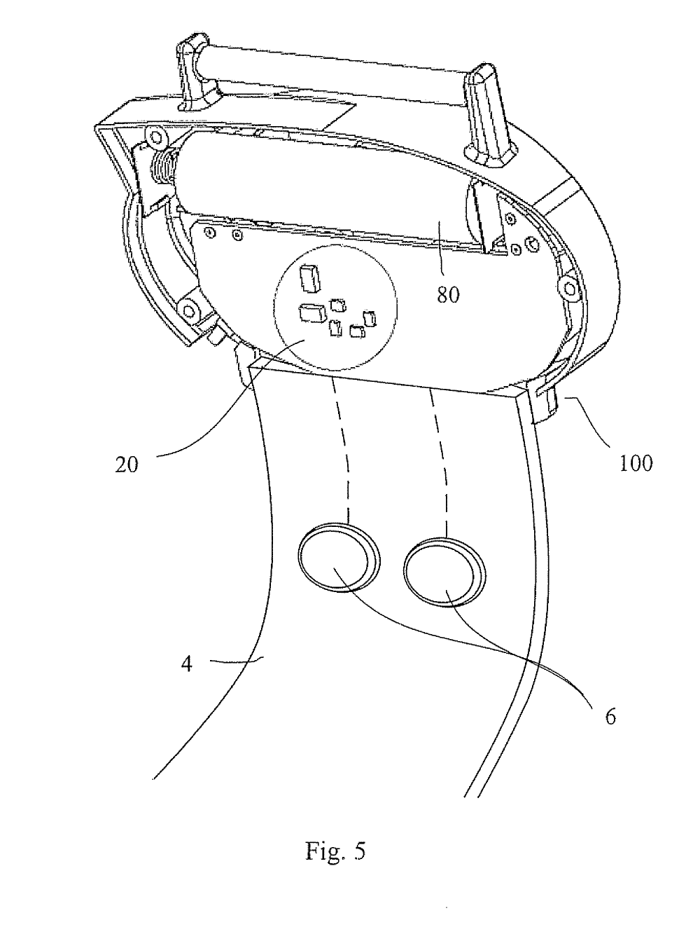

The wristband 100 has a configuration that is wearable on the user's wrist or other limb. The casing 2 is preferably made of ABS plastic. The band 4 has an inner surface 5 and an outer surface 7 and is adapted to couple to the casing 2. The two electrically conductive electrodes 6 lead from the casing 2, and of which one surface is exposed on the inner surface 5 of the band 4, as shown in FIG. 5.

The band 4 may be made of resilient materials, such as plastic, rubber, or composite materials for providing a suitable resilience to wrap around the limb portion of the user. Preferably, the band 4 is elastic fabric made of 95% polyester and 5% Lycra (a trademark used for a brand of spandex). The elastic fabric may be a plain weft rib knitted structure, knitted by using a tubular knitting machine. The diameter of the fiber may be about 10 microns, and the knitting structure of the fabric may be 16 courses by 20 wales per centimeter.

If desired, fasteners, such as VELCRO®-type fasteners, and buckles may be adapted as accessories of the band 4. For instance, the band 4 may contain two sections, with only one section having the two electrically conductive electrodes 6. The section having the two electrically conductive electrodes 6 may be made adjustable in length by using the fastener, and may be connected to the other section of the band 4 by the buckles. The fastener may be sewed onto the fabric by lock stitches, and the buckles sewed using hand-stitches.

In the embodiment illustrated in FIG. 5, the two electrically conductive electrodes 6 are in the shape of a circular button or disc embedded inside the band 4 with one surface being exposed on the inner surface 5 of the band 4. The electrically conductive electrodes 6 may be made of any conductive material such as metal. In one embodiment of the invention, the electrically conductive electrodes 6 are made of 100% stainless steel, or 100% silver coated 100% polyester. The two electrically conductive electrodes 6 can release electrical stimulations upon the skin surface of the user.

Alternatively, the electrically conductive electrodes may be in the form of wires (not shown) woven onto the elastic fabric of the band 4 to form a conductive fabric. The electrically conductive wires may be sewn onto the fabric to make a conductive fabric using an embroidery machine and cotton sewing threads. The width of the embroidery line may be about 2 mm.

To stimulate the Lung Acupoint, one of the electrically conductive electrodes 6 has to be located at the target Acupoint, such as LU7, and the other electrically conductive electrode 6 may be positioned along the Lung Meridian.

Referring to FIG. 7, the RF receiver 240 is coupled to the MCU 31. The RF receiver 240 receives signals from the RF transmitter 220 of the abdomen belt 1. The output of the RF receiver 240 is electrically communicated with the MCU 31. The MCU 31 generates a high-speed pulse width modulation (PWM) signal, which is transmitted to a voltage booster 40 (Resistor, Inductor, Capacitor circuit) to charge a high voltage capacitor 50 (100V) up to a certain voltage level (from 5V to 80V, depending on the chosen electric stimulation level). The electrical actuator 20 having two PNP and two NPN transistors may be provided to control the energy flow from the high voltage capacitor 50 to the user's skin. The output terminals 21 of the electrical actuator 20 are electrically connected to the electrically conductive electrodes 6.

Button switches, such as the “SET” button 72 and “TEST” button 70, and LEDs, such as red LED 74, yellow LED 76 and green LED 78, may also be provided to directly connect to the MCU 31 as a user interface.

The intensity of electrical stimulations may be adjusted or controlled by the “SET” button 72. By pressing the “SET” button 72, a control signal is sent to the voltage booster 40 and the energy is stored in the high voltage capacitor (100V) 50. The stimulation intensity is adjusted by controlling the voltage stored at the high voltage capacitor 50. When the capacitor voltage is charged to the preset voltage level, the MCU 31 will stop the voltage booster 40 from further charging the capacitor 50 and the capacitor 50 is now ready to release energy to the electrical actuator 20. The actuator 20 is composed of an electric circuit, which is able to release electrical pulses in both directions to form biphasic pulses (current flow from electrode No. 1 to electrode No. 2 or from electrode No. 2 to electrode No. 1). The electrical actuator 20 delivers gentle electrical pulses to the skin surface of the user via the electrically conductive electrodes (electrodes Nos. 1 and 2) 6, which are embedded in the band 4.

The LEDs of the wristband 100 may be used to display an event log. For example, the wristband 100 may be programmed so as to indicate the average number of apnea events per hour as follows: Red light: number of apnea per hour is more than 30; yellow light: number of apnea per hour is less than 30 but more than 15; green light: number of apnea per hour is less than 15. This record may be cleared after pressing either the “SET” button 72 or “TEST” button 70 and a new cycle will start.

The abdomen belt 1 and the wristband 100 may be powered by a single AAA size 1.5V battery 80. A DC/DC converter 90 (ST5R50M from STMicroelectronics) may be used to step up voltage up to 5V for the whole circuit.

RF transmitter 220 and RF receiver 240 are commercially available. RF transmitter and RF receiver are illustrated here, although other wireless means based on WiFi, Bluetooth and other short distance communication technologies such as Ultra-Wideband (UWB) technology may also be used.

In use, the user wraps the elastic belt 1 around the abdomen or thorax and then wraps the wristband 100 around the wrist before going to bed. The anti-sleep apnea device helps the user identify the occurrence of sleep apnea during sleep and responds accordingly to the sleep apnea events need in the case of sleep apnea episode in order to avoid prolonged deoxygenation while not fully arouse the user. The anti-sleep apnea device is aimed to provide “symptomatic management” to sleep apnea patients, that is, to detect and respond to the symptoms. On the other hand, a “detect and respond” mechanism detects abnormal signals and provides immediate stimulation.

To illustrate usage of the invention, the exposed surfaces or terminals of the two electrically conductive electrodes 6 on the band 4 are to be positioned at the Lieque acupoint (LU 7), and the band 4 is then tightened to secure the electrodes 6 at the LU 7 location so that the terminals of the electrically conductive electrodes 6 will not slide off the acupoint while moving about in sleep.

The power switch 82 can be shifted to an “ON” position to turn on the anti-sleep apnea device. When the anti-sleep apnea device is turned on, the Green LED 78 will be on. The intensity of electrical stimulation can be set at a desired level by pressing the “SET” button 72, or the anti-sleep apnea device is ready for use. If the “TEST” button 70 is pressed, the LEDs indicating the set stimulation intensity will be blinking and the electrical stimulation will be released. After the electrical stimulation, the anti-sleep apnea device is in operation using the previously stored electric stimulation level.

To set the level of electrical stimulation, simply press the “SET” button 72. By continuously pressing the “SET” button 72, the LEDs will be lightened up in a sequence of green, yellow and red, which represents low, medium and high stimulation intensity, respectively. For example, if the “SET” button 72 is pressed, the green LED 78 will be on to indicate the device is at level 1 (weakest). By pressing the “SET” button 72 again, the yellow LED 76 will be on to indicate level 2. By pressing the “SET” button 72 again, the red LED 74 will be on to indicate level 3 (strongest). It will return to level 1 if the “SET” button 72 is pressed further. The LEDs will automatically switch off after a particular setting is selected and the device is ready to use.

To experience the level of electrical stimulation, the user may press the “TEST” button 70. The user should choose the highest, tolerable intensity of electrical stimulation to achieve a maximal, anti-sleep apnea effect. After testing the intensity of the electric stimulation, the LEDs will be off and the device is in operation and the electric stimulation level selected is automatically stored.

A user who cannot experience or feel an electrical stimulation may need to use the highest level and/or adjust the band 4 tension to ensure the electrodes 6 have good contact with the user's skin. The battery may also need to be replaced if an electrical stimulation is still not experienced.

When in operation mode, the anti-sleep apnea device of the present invention detects the movements or changes in circumference of the abdomen or thorax of the user. The red LED 74 will be on when sleep apnea is detected.

Once the anti-sleep apnea device is in operation, it will count the number of activations (number of times it sends an electric pulse to the user's acupoint). The anti-sleep apnea device will show the number of activations through the LEDs. This counter will be reset to zero after the user presses the “SET” button 72 again. When put the above device to the test, it was confirmed that the smart fabric material, i.e., the fabric strain sensor, is effective in directing the occurrence of apnea. In the experiment, the mean absolute amplitude of the thoracic signal, the abdominal signal and both signals generated from the fabric strain sensor were analyzed when there was an occurrence of apnea and when there was no apnea occurrence. The results showed that there was a significant change of the thoracic signal, the abdominal signals and a mixture of both signals during the occurrence of apnea. The Receiver Operating Characteristic Curve (ROC) Analysis was performed and the result also supported that abdominal signal can be used to detect the occurrence of apnea accurately with a value of 88%.

Table 1 and Table 2 show the effects of meridian point stimulation on alleviating apnea. There were 8 subjects in the meridian group, 5 subjects in the sham group and 7 subjects in the control group. All subjects were requested to stay two nights in a sleep laboratory. The first night was the baseline study. In the second night, electrical stimulation was applied upon the meridian point for the meridian group and the non-meridian point for the sham group respectively during apnea while no electrical stimulation was applied for the control group. The results showed that for the meridian group, there was a decrease of 30% in the Average Apnea Hypopnea Index or AHI (Table 1) and there was also a significant decrease in Average Apnoeic Duration or AAD (Table 2).

TABLE 1
Average Apnea Hypopnea Index - AHI (Lower is better)

| | Average AHI | | |
|---|---|---|---|
| | Baseline | Treatment | % Change |
| Meridian Group | 41.4 | 29.4 | −29.9 |
| Sham Group | 39.2 | 44.1 | +12.5 |
| Control Group | 32.6 | 31.0 | −4.9 |

TABLE 2
Average Apnoeic Duration - AAD (secs) (Lower is better)

| | Average AAD | | |
|---|---|---|---|
| | Baseline | Treatment | % Change |
| Meridian Group | 26.0 | 23.5 | −9.6 |
| Sham Group | 30.3 | 29.2 | −3.6 |
| Control Group | 26.8 | 27.3 | +1.9 |

In the foregoing detailed embodiment of the present invention, it includes a fabric strain sensor attached to and stretchable along with an elastic abdomen belt for detecting occurrence of apnea and automatically starting the electrical stimulation on the acupoint to relieve apnea. However, such automatic detection feature using a fabric strain sensing belt is optional. An anti-apnea device of the present invention may achieve satisfactory results without a fabric strain sensing belt for automatic detection. Instead, when using a device having no automatic detection feature, the user can turn on the device via a switch.

Any patents or publications mentioned in this specification are indicative of the levels of those skilled in the art to which the invention pertains. Further, these patents and publications are incorporated by reference to the same extent as if each individual publication was specifically and individually indicated to be incorporated by reference.

While the present invention has been shown and described with particular references to a number of preferred embodiments thereof, it should be noted that various other changes or modifications may be made without departing from the scope of the present invention.

What is claimed is:

1. An anti-sleep apnea device comprising:
(a) a pair of electrodes, one of which is adapted to be disposed on a user's limb for releasing electrical pulses to an acupoint of said user;
(b) a control unit to which said electrodes are connected, said control unit comprising an electrical actuator for generating said electrical pulses; and
(c) a switch for turning on or off said control unit.

2. The anti-sleep apnea device according to claim 1, further comprising:
(d) fabric strain sensor adapted to be disposed on a user's abdomen, thorax or both and to detect a change of circumference of the abdomen, thorax or both;
(e) a signal-processing unit for processing signals from said fabric strain sensor in response to the change of circumference of the user's abdomen, thorax or both; and
(f) a transmitting unit for transmitting signals from said first signal-processing unit to said control unit for turning on or off said electrical actuator.

3. The device as claimed in claim 2 wherein, said fabric strain sensor is provided on an elastic belt adapted to be wrapped around the user's abdomen or thorax.

4. The device as claimed in claim 2 wherein, said electrodes are provided on a wristband adapted to be wrapped around the user's wrist.

5. The device as claimed in claim 2, wherein said signal-processing unit comprises an amplifier/signal conditioner and a first micro-control unit.

6. The device as claimed in claim 5, wherein said first micro-control unit comprises a software algorithm to measure said signals from said fabric strain sensor.

7. The device as claimed in claim 2, wherein said control unit comprises a second micro-control unit and an electrical actuator.

8. The device as claimed in claim 7, wherein said control unit further comprises a high voltage capacitor coupled to said electrical actuator, and a voltage booster for charging said high voltage capacitor.

9. The device as claimed in claim 2, wherein said transmitting unit is RF transmitter and RF receiver.

10. The device as claimed in claim 2, wherein said acupoint is an acupoint along Lung meridian.

11. The device as claimed in claim 10, wherein said acupoint is LU7 (Lieque) located around the wrist of the user.

12. The device as claimed in claim 1, further comprising a set button for presetting said electrical pulses at a preset level of intensity.

13. The device as claimed in claim 1, further comprising a LED for displaying said preset level of intensity, or displaying events of releasing said electrical pulses, or both.

14. The device as claimed in claim 1, further comprising a test button for testing said preset level of intensity of said electrical pulses.

15. The device as claimed in claim 1, wherein the electrodes are made of stainless steel or silver coated polyester.

16. A method for reducing sleep apnea comprising the steps of:
(a) detecting signals of resistance change generated by a fabric strain sensor in response to the movement of the abdomen, thorax or both of a user; and
(b) transmitting stimulation signals in response to the signal change of said fabric strain sensor to two pulse-releasing electrodes thereby stimulating an acupoint of the user.

17. The method as claimed in claim 16, wherein said step (a) further comprising the steps of preprocessing said signals of said fabric strain sensor by an amplifier/signal conditioner, and interpreting said preprocessed signals by a first micro-control unit.

18. The method as claimed in claim 17, wherein said interpretation of said preprocessed signals comprising the step of calculating an index which indicates the amplitude change or the standard deviation change of the said signals.

19. The method as claimed in claim 18, further comprising the steps of processing said signals from said first micro-control unit, and releasing stimulation request signals to a second micro-control unit to release electrical stimulation by an electrical actuator when the reduction of said index is over a pre-defined threshold.

20. The method as claimed in claim 19, further comprising the steps of providing a high voltage capacitor for storing electrical charges and releasing energy to said electrical actuator, and providing a voltage booster for charging said high voltage capacitor.

21. The method as claimed in claim 16, wherein said step (b) further comprising transmitting said signals from said first micro-control unit to said second micro-control unit by a wireless means.

22. The method as claimed in claim 15, wherein said acupoint is an acupoint along Lung meridian.

23. The method as claimed in claim 22, wherein said Lung Acupoint is LU7 (Lieque) located around the wrist of the user.

24. The method as claimed in claim 16, wherein said fabric strain sensor is provided on an elastic belt adapted to be wrapped around the user's abdomen, thorax or both.

25. The method as claimed in claim 16, wherein said electrodes are provided on a wristband adapted to be wrapped around the user's wrist.
